(19) United States
(12) Patent Application Publication
(10) Pub. No.: US 2016/0081941 A1
(43) Pub. Date: Mar. 24, 2016

(54) PHARMACEUTICAL COMPOSITION WITH CONTROLLED RELEASE OF METABOLICALLY ACTIVE SUGAR

(71) Applicants: FAKUTNÍ NEMOCNICE HRADEC KRÁLOVÉ, Hradec Králové (CZ); VETERINÁRNÍ A FARMACEUTICKÁ UNIVERZITA BRNO, Brno (CZ)

(72) Inventors: David NEUMANN, Hradec Králové (CZ); Ales FRANC, Brno (CZ); Jan MUSELIK, Brno (CZ)

(73) Assignees: FARMACEUTICKÁ UNIVERZITA BRNO, Brno (CZ); FAKULTNÍ NEMOCNICE HRADEC KRÁLOVÉ, Hradec Kráové (CZ)

(21) Appl. No.: 14/889,801

(22) PCT Filed: Jan. 13, 2014
(86) PCT No.: PCT/CZ2014/000004

§ 371 Date: Nov. 6, 2015

(30) Foreign Application Priority Data
May 9, 2013 (CZ) PV2013-338

(51) Int. Cl. A61K 9/50; A61K 47/10; A61K 47/02; A23L 1/09; A61K 47/32; A61K 47/36; A61K 47/12; A23L 1/00; A61K 31/7004; A61K 47/38

(52) U.S. Cl. CPC A61K 9/5047; A61K 31/7004; A61K 9/5026; A61K 47/10; A61K 47/02; A61K 47/38; A61K 47/32; A61K 47/36; A61K 47/12; A23L 1/0047; A23L 1/09; A23V 2002/00

(57) ABSTRACT

Pharmaceutical composition with a controlled release of a metabolically active sugar, especially glucose, consisting of pellets containing a pellet core and a pellet core coating, the substance of which consists in that the pellet core contains at least 50% by weight of the metabolically active sugar, related to the total weight of the pellet core, at least one osmotically active substance or a disintegrant in a total amount up to 50% by weight, related to the total weight of the pellet core, and possibly standard excipients, wherein the pellet core coating consists of a polymeric membrane comprising a polymer of a cellulose derivative and/or methacrylate derivative, the weight of the pellet core coating being 5-50% by weight, related to the total weight of the pellet and the pharmaceutical composition contains pellets with at least one of the four different release profiles of the metabolically active sugar.

### FIELD OF THE INVENTION

This invention concerns pharmaceutical compositions with a controlled release of metabolically active sugar.

### BACKGROUND OF THE INVENTION

Diabetes mellitus (DM) is caused by a heterogeneous group of chronic progressive disorders characterized by insulin secretion or function disorders and consequent metabolic changes, which lead to the onset of clinical symptoms and acute and chronic complications (Craig, M. E., Hattersley, A., Donaghue, K. C.: Definition, Epidemiology and Classification of Diabetes Mellitus. Paediatric Diabetes, 2006; 7: 343-351; ISPAD Consensus Guidelines 2006-9009. Moderní d\check{e}tská diabetologie. Galén, Prague 2009: 286 s). Etiologically, there is a prevalence of type 2 diabetes caused by insulin resistance and gradually progressing disorder of the beta cells function. It is often connected with the metabolic syndrome. Diabetes mellitus type 1 is caused by an autoimmune damage to beta cells in the pancreatic islets of Langerhans (autoimmune insulitis). An absolute lack of insulin leads to the clinical picture of diabetes (Neumann, D. Diabetes mellitus 1. typu u d\check{e}tí In Bayer M. (ed.) Pediatrie. Triton, Prague 2011: 350 s.) In children's population, this causes over 97% of diabetes cases. The Czech Republic ranks among countries with medium incidence; based on the data as of 2003 approx. 18.5/100000 children up to 15 years of age per year, i.e. 250-300 new cases per year. The prevalence totals 1.01/1000 children up to 14 years.

Hypoglycemia is an acute complication in the diabetes mellitus type 1 and 2 therapy. It develops during the therapy by insulins or oral antidiabetics (PAD) and in extreme cases it threatens the patient's life. Furthermore, its solving by the construction of insulin analogues can be insufficient in situations when a patient cannot eat. Patients often solve their fear of hypoglycemia by an unreasonable intake of saccharides, which results in hyperglycemias and the development and progression of chronic complications. The drug form with a controlled release of glucose compensates hypoglycemia and allows decreasing of the dose of saccharides, which are taken by patients “preventively”. Typical examples are nocturnal hypoglycemia, hypoglycemia during physical activities of children during team sports, hypoglycemia in preschoolers and school children due to an imbalance between the applied insulin, energy expenditure and the amount of consumed saccharides.

The drug form containing glucose releases specific amounts of glucose necessary to maintain its sufficient levels at required intervals, which allows to balance the effect of insulin or PAD. It can be administered with an adequate advance prior to the critical moment at a time, which is suitable and convenient. This improves patients' compliance. The drug form with a controlled release of glucose provides a real opportunity to prevent hypoglycemia in type 1 and 2 diabetics and also to prevent undesired “preventive” high intake of saccharides. This drug form reduces the infringement of usual patient's activities and therefore improves patients' compliance. It is especially suitable for the prevention of nocturnal hypoglycemia, hypoglycemia during an increased physical strain (e.g. sport activities) and for the prevention of hypoglycemia in drivers. This drug form mechanism is based on a release of a defined amount of glucose at defined time intervals. It also offers a suitable solution for other groups of people, e.g. patients with particular metabolic disorders, high-endurance sportsmen and in situations, when it is not possible to interrupt a particular activity for several hours.

Pharmacotherapy: Type 1 diabetes therapy employs insulin and its analogues. Both insulin and analogues are protein macromolecules consisting of two protein chains, which have together 51 amino acids. Based on the effect duration, there are very short-acting insulin analogues (effect 2-5 hours), human insulin (effect 6 hours), intermediate-acting insulins (NPH insulin acting 12-18 hours) and long-acting insulin analogues (effect 24-36 hours). Pharmacotherapy of type 2 diabetes also includes various groups of oral antidiabetics with variably long and sometimes prolonged effect.

Hypoglycemia: Develops due to an imbalance between the applied insulin (or the effect of oral antidiabetics), energy expenditure and the amount of consumed saccharides. Most hypoglycemias can be tracked down because they are caused by incorrect therapeutic decisions (Clarke, W., Jones T, Rewers, A., et al. Assessment and Management of Hypoglycaemia in Children and Adolescents with Diabetes. Pediatric Diabetes 2008; 9: 165-174). However, some hypoglycemias are connected with the existing therapy potential and they can rarely be solved by a daily routine adjustment, usually disturbing the patient's ordinary life. These conditions include especially nocturnal hypoglycemia caused by the maximum effect of insulin in the time with physiologically lowest consumption (the Somogyi effect). Then, there is hypoglycemia during sport activities, which cannot be interrupted at regular intervals (team sports) and hypoglycemia in small children, who eat reluctantly and irregularly.

The risk of hypoglycemia connected with the above situations can easily be influenced by the application of drug forms containing glucose released at a desired time. This prevents the drop of glycemia below 4 mmol/l, compensating the effect of insulin/drug at the time of its maximum effect or activity/impossibility to eat.

Therefore, the benefit of a controlled release drug form lies in the broadening of therapeutic spectrum used in the treatment of diabetes. This therapy decreases the number of interventions into a usual life of people with diabetes, with a special focus on child patients. The design of this drug form is based on practical needs of patients to keep their eventful and variable modern lifestyle. It facilitates the possibility of a personalized diet in the form of food supplements for vulnerable population of patients. Furthermore, costs on its use are very low compared with other components of a diabetic patient treatment. If patients are properly informed, there can be expected a reduction of hypoglycemias as well as prevention of hyperglycemias, which occur due to attempts to prevent hypoglycemia. Its appropriate use can help improve the quality of patients' life and probably even slow down chronic and prevent acute complications.

A search of scientific literature implies that to date, there has not been any satisfactory solution offering a drug form facilitating controlled release of glucose. An attempt to solve this situation came from a food company the Estee Corporation in 1994, which submitted a patent application to register a food supplement in the form of microparticles containing saccharides, which are gradually released and modify blood sugar levels (The Estee Corp. U.S. Pat. No. 5,545,410. Priority: 21 Oct. 1994). However, it has not been implemented in the market yet. The offered solution lies in a controlled release of glucose without any lag time, during which the release would be either none or minimal. Similarly, a patent application from 1995 by Hercules Inc. claims a special preparation of food utilizing a controlled glucose release. However, it does not concern any defined drug form and it did not offer a profile with a required lag time (Hercules Inc. U.S. Pat. No. 5,795,606. Priorita 2 Oct. 1995).

It can be therefore stated that a drug form, which enables controlled release of glucose as a food supplement, has not been available to date and this problem has not been solved. Milojevic et al. describe another type of a controlled or delayed release of glucose from particle systems. Nevertheless, they used glucose as a model drug for the transport of an active substance into the large intestine utilizing pellets coated with a mixture of water insoluble ethyl cellulose polymer with amylose. Their aim was to achieve a release of the drug in the area of colon, large intestine, which is virtually useless for our purpose from the perspective of a time delay (Milojevic, C. et al. Amylose as a coating for drug delivery to the colon: Preparation and in vitro evaluation using glucose pellets. Journal of Controlled Release 1996; 38; 85-94).

### SUMMARY OF THE INVENTION

In this invention, the metabolically active sugar means glucose or such saccharides, which metabolize into glucose in human body. They can be monosaccharides, disaccharides or polysaccharides.

A subject matter of this invention is a drug form containing microparticles or pellets with a controlled release of metabolically active sugar. The drug form consists of an inert core in the form of a pellet containing metabolically active sugar, and a membrane coating, which releases glucose at particular intervals during its passage through the GI tract. Pellets are consequently mixed with semisolid or liquid food or dispersed in a drink and consumed with food. The resulting preparation consists of one or more types of pellets differing in color coating, which release glucose at different time intervals. This helps achieve safe levels of blood sugar for a required time (e.g. night) without disturbing the diabetic's daily routine. This drug can also be used during an increased physical strain in sportsmen and hypoglycemias in preschoolers or school children in the morning, when there can be an insufficient food intake. It is also possible to use it in drivers with diabetes, high-endurance sportsmen, in the prevention of nocturnal hypoglycemias or other medical conditions, e.g. people with inherited metabolic disorders. A diabetic patient prepares the required mixture on their own, based on the specificity of their therapy and their activities.

In total, there are four types of pellets differing in color. Their initial letter indicates the release rate:
R (rapid release rate) pellets are pink and they release max. 10% within 60 minutes and min. 70% within 120 minutes.
M (medium release rate) pellets are blue and they release max. 10% within 60 minutes; max. 30% within 120 minutes and min. 70% within 360 minutes.
D (delayed release rate) pellets are green and they release max. 30% within 60 minutes; 30-50% within 270 minutes and min. 70% within 540 minutes.
S (slow release rate) pellets are orange and they release max. 10% within 120 minutes; max. 30% within 270 minutes and min. 70% within 540 minutes.

However, these colors are facultative and given only as examples. The subject matter of this patent stipulated herein is not limited by them and any other colors can be used in their production because the surface color has no impact on the release rate of metabolically active sugar.

The pellet consists from a core formed by the extrusion-spheronization method and from a coating by polymeric membrane. The core contains at least 50% of glucose and at least one pharmaceutically acceptable excipient or their mixture, which adjusts the release rate based on various mechanisms, at a concentration 1-50% relating to the core weight. These excipients include microcrystalline cellulose (commercially manufactured as the Avicel PH-line, for example), which forms the main filling, facilitates good spheronization and creates capillary structure ensuring penetration of water into a pellet's core (which allows dissolving of metabolically active sugar and its release). Suitable excipients include swelling substances, which cause osmotic pressure on the internal part of the membrane and facilitate accelerated release of a substance through the membrane, or they cause the membrane rupture and consequently dissolving or release of metabolically active sugar. These substances include e.g. carboxymethyl cellulose sodium salt or its cross-linked form (commercially available as Ac-Di-Sol, for example) or directly a mixture of microcrystalline cellulose and carboxymethyl cellulose sodium salt (commercially available as Avicel RC-line, for example); then there is carboxymethyl starch (CMS), cross-linked polyvinylpyrrolidone (Crosspovidone) or pregelatinized starch. Substances, which increase osmotic pressure, also include compounds from the group of soluble polymers, e.g. polyethylene glycols with molecular weight 400-8 000 Da, urea or inorganic salts such as sodium chloride or potassium chloride. The group of excipients includes substances in the form of acids or alkali, whose mixtures, usually in stochiometric ratio, react upon contact with water, which penetrates the pellet and release carbon dioxide. These are disintegrants, whose mechanism is based on the formation of gasses. This expands the pellet or increases its volume, which leads to the membrane rupture and dissolving/release of the metabolically active sugar contained in the core. This group usually includes organic acids such as citric, succinic, malic and tartaric acid, etc., and alkali in the form of sodium or potassium carbonate, hydrogen carbonates, phosphates or hydrogen phosphates. Membrane has the form of a film coating and it is applied on the core. It prevents the release of metabolically active sugar in the first phase after the administration and creates the required lag time. After the consequent activation of osmotically active substances or disintegrants, membrane breaks up or even falls off, which accelerates dissolving and releasing of metabolically active sugar into the body. The membrane can be semipermeable and insoluble from e.g. ethyl cellulose (EC), or soluble, from hydroxypropyl methylcellulose (HPMC) or carboxymethylcellulose sodium salt (Na CMC). There can also be used cellulose polymers in the form of esters of inorganic acids, which can dissolve due to the passage into neutral or alkaline GI tract environment in the area of small intestine, which initiates dissolving and releasing of metabolically active sugar. This group of esters includes hydroxypropyl methylcellulose phthalate (HPMCP), hydroxypropyl methylcellulose acetate (HPMCA), hydroxypropyl methylcellulose acetate succinate (HPMCAS) and hydroxypropyl methylcellulose succinate (HPMCS). Other membrane-forming polymers include copolymers of methacrylic acid, methyl methacrylic acid; polymethacrylic and ethylacrylic acid in various combinations and ratio. These compounds are commercially known under the brand Eudragit, namely Eudragit E, which dissolves in acidic environment; and Eudragit L, S and FS, which dissolve in neutral or alkaline environment and which can dissolve due to the passage into neutral or alkaline GI tract environment in the area of small intestine. This initiates dissolving and releasing of metabolically active sugar. Then there are Eudragit RL, RS, NE and NM, which can be used for gradual release.

Films from individual membrane-forming polymers can be applied in the form of organic solutions or aqueous dispersions. All the above listed film-forming compounds can be applied in a form of true or colloidal solutions in ethanol, methanol, acetone, isopropanol or their mixtures in an arbitrary order and ratio, with a possible adding of water up to 50% of the total mixture weight. Then they can be applied in a form of in situ prepared aqueous dispersions; it is possible to utilize commercially available aqueous dispersions as semi-products to prepare coating dispersions. These include dispersions containing acrylate (Eudragit L 30 D; L 55 30 D; RS 30 D; RL 30 D; FS 30 D; NM 30 D; NE 30 D; NE 40 D) or ethyl cellulose (Aquacoat ECD, Surelease, Acryl-EZE, Nutrateric). These mixtures are then mixed with plasticizers in the form of esters of polyprotic acids such as dibutyl subacetate, triethyl citrate, diethyl citrate or polyhydric alcohols such as glycerol, propylene glycol, polyethylene glycol, etc., or polyols and sugars such as lactose. These dispersions can also contain surfactants in a form of tensides, most commonly sodium lauryl phosphate and polysorbate, and dyes or pigments. There can be possibly contained anti-adhesive agents such as talcum powder or magnesium stearate and anti-foaming agents such as silicon emulsion (Simethicon). The total weight of the coating ranges from 5 to 50% of the total weight of the coated pellet.

This coating can be further covered by a pigmenting hydrophilic film formed by a mixture on the base of hydrophilic polymer, plasticizer or tensid and a required pigment (preferably pink, orange, green or blue). Hydrophilic polymers are usually hydroxypropyl methylcellulose (HPMC), methylcellulose (MC), polyvinyl alcohol (PVA) and polyvinylpyrrolidone (PVP) or their mixture at an arbitrary ratio. These mixtures are then mixed with plasticizers in the form of esters of polyprotic acids such as dibutyl subacetate, triethyl citrate, diethyl citrate or polyhydric alcohols such as glycerol, propylene glycol, polyethylene glycol, etc., or polyols and sugars such as lactose. These pigmenting dispersions can also contain surfactants in a form of tensides, most commonly sodium lauryl phosphate and polysorbate, and dyes or pigments. There can be possibly contained anti-adhesive agents such as talcum powder or magnesium stearate, and anti-foaming agents such as silicon emulsion (Simethicon). Pigmenting films constitute 0.5-5% of the total weight of the coated pellet. Commercially, these compounds in the form of mixtures for the preparation of coating aqueous dispersions are known under the mark OPADRY and OPADRY II or SEPIFILM and SEPICOAT. These films dissolve within 5 minutes after the contact with digestive juices and they do not have any impact on the release of metabolically active sugar.

Pellet cores or uncoated pellets are made by the extrusion method, i.e. a suitable excipient or a mixture of suitable excipients from the above list (in this case metabolically active sugar) is added to the active substance, then a suitable wetting agent is added to the mixture, usually water or solutions of binding agents (if binding agents have not already been included in the solid part) and again mixed together. The wet matter is then processed in the extruder consisting of a feed screw and a sieve with round holes. The wetted matter is compressed and thickened by the feed screw and passed through the round sieve with a suitable holes size, which leads to the formation of cylindrical granules, the so-called “spaghetti”. The second part of the process is executed in a spheronizer, i.e. a cylindrical container with a spinning plate on the bottom, whose surface is fitted with friction pattern with sharp edges. Granules, which fall on the spinning plate, are cut by the sharp edges into segments with a length ranging from 0.1-1.5 mm, corresponding to the spaces between sharp edges. These segments are consequently sent by centrifugal force to the outside wall of the cylindrical container. The rotary action of the moving material causes that they are converted into spheres of a desired size, corresponding to the spaces between sharp edges. Final pellets are then dried and further processed.

An alternative method is a rotary agglomeration, where pellets are processed directly in a fluid device, i.e. spheronized by centrifugal force on the spinning plate, polymer-coated and dried. Pellets are coated by solutions or dispersions in a cylinder coating device, fluid bed device by top, tangential or bottom-spray, or in the Wurster coater. Pellets coated by ethyl cellulose-based water insoluble films are then heat treated to achieve even glassy surface facilitating a standard drug release. They are usually heated in a hot-air drier at 50-60° C. for 45-60 minutes. The estimated dose of metabolically active sugar in pellets will correspond to approximately 10 g of glucose (in diabetology one replaceable unit); the weight of the drug form with this amount of glucose is 15-40 g. As a result, patients can prepare necessary mixture of pellets according to their colors and therapeutic needs.

### EXAMPLES

### Example 1

### Core Composition

| Component | Amount (g) |
|---|---|
| 1. Glucose | 50.0 |
| 2. Avicel PH 101 | 50.0 |
| 3. Water | 45.0 |

### Working Procedure:

Components 1-2 are weighed;
The mixture is stirred for 1 minute in a high-speed mixer at 400 rpm;
Water is added and the mixture is stirred for 1 more minute at 400 rpm;
The mixture is extruded through the extrusion screen with 1.00 mm holes;
The extrudate is spheronized for 1 minute at 1 000 rpm;
Resulting pellets are dried for 4 hours at 50° C.

### Composition of the Coating Dispersion:

| Components | Amount (g) |
|---|---|
| 1. Eudragit L 12.5 | 94.5 |
| 2. Triethyl citrate | 2.5 |
| 3. Water | 3.0 |
| 4. Isopropanol | 37.0 |
| 5. Magnesium stearate | 7.0 |

The components 1-4 are stirred for 30 minutes by a slow-speed stirrer;
Magnesium stearate is added; the mixture is stirred for 5 minutes and used for spraying.

### Working Procedure:

The total of 100.0 g of cores are added into the chamber of a fluid coating device and spraying starts under the following conditions:

| | |
|---|---|
| Spraying speed (g/ml) | 7.0 |
| Air pressure (bar) | 0.75 |
| Initial temperature (° C.) | 45 |
| Batch amount (g) | 100.0 |
| Jet diameter (mm) | 1.0 |

After applying the whole amount of coating dispersion, pellets are dried for another 5 minutes.

### Example 2

### Core Composition

| Components | Amount (g) |
|---|---|
| 1. Glucose | 50.0 |
| 2. Avicel PH 101 | 25.0 |
| 3. Avicel RC 591 | 25.0 |
| 4. Water | 40.0 |

### Working Procedure:

Components 1-3 are weighed;
The mixture is stirred for 1 minute in a high-speed mixer at 400 rpm;
Water is added and the mixture stirred for 1 more minute at 400 rpm;
The mixture is extruded through the extrusion screen with 1.00 mm holes;
The extrudate is spheronized for 1 minute at 1 000 rpm;
Resulting pellets are dried for 4 hours at 50° C.

### Composition of the Coating Dispersion

| Components | Amount (g) |
|---|---|
| 1. Aquacoat ECD | 267.0 |
| 2. Dibutyl subacetate | 20.0 |
| 3. Water | 55.0 |

The components 1-3 are stirred for 30 minutes by a low-speed stirrer and the mixture is used for spraying.

### Working Procedure:

The total of 100.0 g of cores are added into the chamber of a fluid coating device and spraying starts under the following conditions:

| | |
|---|---|
| Spraying speed (g/ml) | 10 |
| Air pressure (bar) | 2 |
| Initial temperature (° C.) | 60 |
| Batch amount (g) | 100.0 |
| Jet diameter (mm) | 1.0 |

After applying the whole amount of coating dispersion, pellets are dried for another 60 minutes at 50° C. in a hot-air oven.

### Example 3

### Core Composition

| Components | Amount (g) |
|---|---|
| 1 Glucose | 50.0 |
| 2 Avicel PH 101 | 47.0 |
| 3 CMS | 3.0 |
| 4 Water | 40.0 |

### Working Procedure:

Components 1-3 are weighed;
The mixture is stirred for 1 minute in a high-speed mixer at 400 rpm;
Water is added and the mixture is stirred for 1 more minute at 400 rpm;
The mixture is extruded through the extrusion screen with 1.00 mm holes;
The extrudate is spheronized for 1 minute at 1 000 rpm;
Resulting pellets are dried for 4 hours at 50° C.

### Composition of the Coating Dispersion

| Components | Amount |
|---|---|
| 1 Aquacoat ECD | 133.5 |
| 2 Dibutyl subacetate | 10.0 |
| 3 Water | 22.5 |

The components 1-3 are stirred for 30 minutes by a low-speed stirrer and use the mixture for spraying.

### Working Procedure:

The total of 100.0 g of cores is added into the chamber of a fluid coating device and spraying starts under the following conditions:

| | |
|---|---|
| Spraying speed (g/ml) | 10 |
| Air pressure (bar) | 2 |
| Initial temperature (° C.) | 60 |
| Batch amount (g) | 100.0 |
| Jet diameter (mm) | 1.0 |

After applying the whole amount of coating dispersion, pellets are dried for another 60 minutes at 50° C. in a hot-air drier.

### Example 4

### Core Composition

| Components | Amount (g) |
|---|---|
| 4 Glucose | 50.0 |
| 5 Avicel PH 101 | 44.0 |
| 6 Citric acid | 2.73 |
| 7 Sodium bicarbonate | 3.27 |
| 8 Ethanol | 40.0 |

### Working Procedure:

Components 1-4 are weighed;
The mixture is stirred for 1 minute in a high-speed mixer at 400 rpm;
Water is added and the mixture is stirred for 1 more minute at 400 rpm;
The mixture is extruded through the extrusion screen with 1.00 mm holes;
The extrudate is spheronized for 1 minute at 1 000 rpm;
Resulting pellets are dried for 4 hours at 50° C.

### Composition of the Coating Dispersion

| Components | Amount (g) |
|---|---|
| 1 Aquacoat ECD | 133.5 |
| 2 Dibutyl subacetate | 10.0 |
| 3 Water | 22.5 |

The components 1-3 are stirred for 30 minutes by a low-speed stirrer and the mixture is used for spraying.

### Working Procedure:

The total of 100.0 g of cores are added into the chamber of a fluid coating device and spraying starts under the following conditions:

| | |
|---|---|
| Spraying speed (g/ml) | 10 |
| Air pressure (bar) | 2 |
| Initial temperature (° C.) | 60 |
| Batch amount (g) | 100.0 |
| Jet diameter (mm) | 1.0 |

After applying the whole amount of coating dispersion, pellets are dried for another 60 minutes at 50° C. in a hot-air drier.

### Example 5

### Coating of Pellets by Pigmenting Dispersion

### Composition of the Coating Dispersion

| Components | Amount (g) |
|---|---|
| 1 OPADRY II | 5.0 |
| 2 Water | 45.0 |

OPADRY is supplied in various colors.

During intensive stirring Opadry is gradually added into the water tank, directly into the vortex created by the stirrer blades. After its complete disintegration, the rotation is decreased to minimum speed, which still ensures stirring of the whole batch. Then stirring continues for 45 minutes and after this the dispersion is used for spraying.

### Working Procedure:

The total of 100.0 g of cores are added into the chamber of a fluid coating device and start spraying under the following conditions:

| | |
|---|---|
| Spraying speed (g/ml) | 10 |
| Air pressure (bar) | 2 |
| Initial temperature (° C.) | 60 |
| Batch amount (g) | 100.0 |
| Jet diameter (mm) | 1.0 |

The whole amount of coating dispersion is applied.

### Example 6

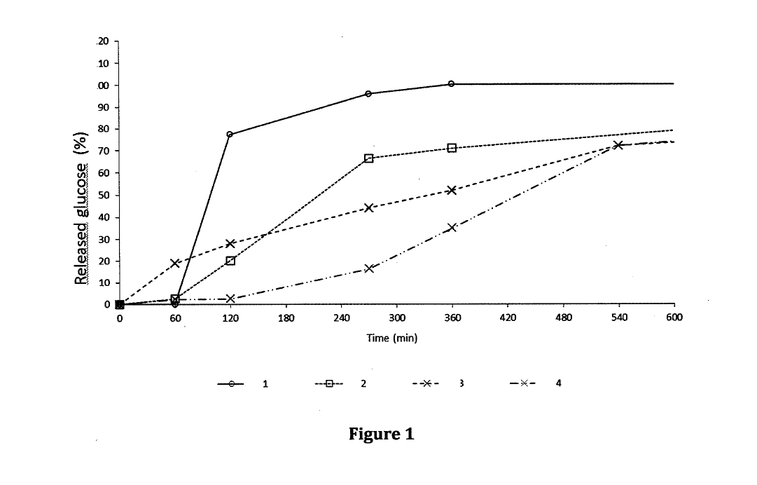

This example illustrates glucose release from individual formulas according to samples 1-4, graphically demonstrated in FIG. 1.

Graphs illustrate the release process in an artificial digestive juice (pH 1.2), which was replaced after 60 minutes by an artificial intestinal juice (pH 6.8). The process was implemented at 100 rpm, paddles method, with off-line taking of samples and HPLC analysis.

1. Pharmaceutical composition with a controlled release of a metabolically active sugar, especially glucose, consisting of pellets containing a pellet core and a pellet core coating characterized in that the pellet core contains at least 50% by weight of the metabolically active sugar, related to the total weight of the pellet core, at least one osmotically active substance or a disintegrant in a total amount up to 50% by weight, related to the total weight of the pellet core, and possibly standard excipients, wherein the pellet core coating consists of a polymeric membrane comprising a polymer of a cellulose derivative and/or methacrylate derivative, the weight of the pellet core coating being 5-50% by weight, related to the total weight of the pellet and the pharmaceutical composition contains pellets with at least one of the four different release profiles of the metabolically active sugar, in frame of which:
it releases 0-10% of the original weight of the metabolically active sugar within 60 minutes after administering and 70-100% of the original weight of the metabolically active sugar within 120 minutes after administering;
it releases 0-10% of the original weight of the metabolically active sugar within 60 minutes after administering, 0-30% of the original weight of the metabolically active sugar within 120 minutes after administering, and 70-100% of the original weight of the metabolically active sugar within 360 minutes after administering;
it releases 0-30% of the original weight of the metabolically active sugar within 60 minutes after administering, 30-50% of the original weight of the metabolically active sugar within 270 minutes after administering, and 70-100% of the original weight of the metabolically active sugar within 540 minutes after administering;
it releases 0-10% of the original weight of the metabolically active sugar within 120 minutes after administering, 0-30% of the original weight of the metabolically active sugar within 270 minutes after administering, and 70-100% of the original weight of the metabolically active sugar within 540 minutes after administering.

2. Pharmaceutical composition according to the claim 1 characterized in that pellets with individual release profiles of the metabolically active sugar differ in their colors.

3. Pharmaceutical composition according to the claim 2 characterized in that the pellets with individual release profiles of the metabolically active sugar differ in their colors due to different pigments contained in the pellet core coating.

4. Pharmaceutical composition according to the claim 2 characterized in that that the pellets with individual release profiles of the metabolically active sugar differ in their colors due to different pigments contained in a hydrophilic polymeric film on the pellet core coating

5. Pharmaceutical composition according to the claim 4 characterized in that the hydrophilic polymeric film consists of hydroxypropyl methylcellulose, methylcellulose; polyvinylpyrrolidone a/or polyvinyl alcohol.

6. Pharmaceutical composition according to the claim 1 characterized in that the polymer of the cellulose derivative, which forms the polymeric membrane that forms the pellet core coating, is a polymer from the group including a polymer of ethyl cellulose, hydroxypropyl methylcellulose, carboxymethylcellulose sodium salt and phthalate, acetate, succinate and hydroxypropyl methylcellulose acetosuccinate.

7. Pharmaceutical composition according to the claim 1 characterized in that the polymer of the methacrylate derivative, which forms the polymeric membrane that forms the pellet core coating, is a polymer from the group including copolymers of methacrylic acid, methyl methacrylic acid, polymethacrylic and ethylacrylic acid.

8. Pharmaceutical composition according to the claim 1 characterized in that the pellet core contains, as osmotically active substance, polyethylene glycol with molecular weight 400 to 8000 Da, urea, mannitol, sorbitol, sodium chloride or potassium chloride.

9. Pharmaceutical composition according to the claim 1 characterized in that the pellet core contains, as disintegrant, carboxymethyl cellulose sodium salt or its cross-linked form, a mixture of microcrystallic cellulose and carboxymethyl cellulose sodium salt, carboxymethyl starch, cross-linked polyvinylpyrrolidone, pregelatinized starch, organic acid such as citric, succinic, malic and tartaric acid, and alkali in the form of sodium or potassium carbonate, hydrogen carbonates, phosphates or hydrogen phosphates.
